(19) United States
(12) Patent Application Publication
(10) Pub. No.: US 2003/0088196 A1
(43) Pub. Date: May 8, 2003

(54) Customized physiological monitor

(75) Inventor: Burton Steve, Midlothian, VA (US)

Correspondence Address: MILLEN, WHITE, ZELANO & BRANIGAN, P.C., 2200 CLARENDON BLVD., SUITE 1400, ARLINGTON, VA, 22201, US

(73) Assignee: EPM Development Systems Corporation, Midlothian, VA

(21) Appl. No.: 09/985,389

(22) Filed: Nov. 2, 2001

(51) Int. Cl. A61B 5/103

(52) U.S. Cl. 600/587

(57) ABSTRACT

A device to be worn by a user when performing therapy or exercise monitors the physiological functions and/or the performance of the exercise by the user. The device provides feedback to the user based on a predetermined exercise program that is contained in the device. The device may be programmed by the user or, alternatively, may be programmed by a trainer or therapist caring for the user.

The present invention relates generally to a physiological monitoring device to be worn by a user when performing therapy or simply training. The device monitors the therapy or training and guides the user through a prescribed exercise program. The device may be programmed by the user or, alternatively, may be programmed by a trainer or by a therapist caring for the user.

### BACKGROUND OF THE INVENTION

There are numerous exercise monitors and exercise aids presently available and known to those in the fitness and rehabilitation industries. Many of these devices attempt to be user-friendly in one or more features. Nevertheless, none is adapted to be programmed or customized for a specific therapy regime or training schedule.

Many existing monitors include preset or arbitrary workouts and/or goals to be attained by the user wearing the monitor. These monitors may detect the extent or amount of exercise accomplished or may monitor physical fitness by way of, for instance, heart rate. The programs offered by these devices, however, are limited to the particular exercises or regimes programmed therein. If a user needs a specific exercise regime that is not programmed into a device, then that device is simply not as effective as possible for that user.

Many existing monitors or exercise aids also have complicated protocols in setting up the device to obtain particular workout parameters. These devices may require the input of all of the requested workout parameters. To the extent that the device may aid a user, there is still substantial information that is typically required to be input.

Conventional monitors are also generally limited in the physical functions that they are able to monitor. For instance, one type of monitor may only detect the amount of exercise accomplished by a user. Other types of monitoring may detect only one or a few physical functions of a user during a workout. These devices include those that detect heart rate. If a specific combination of attributes is desirable to monitor, then existing devices typically are limited in the combination of monitored functions.

Conventional exercise aids are also, for the most part, self-policing. That is, the user may or may not pay attention to the workout suggested by the device. There is no oversight with respect to the compliance of the user in performing the workout or in reviewing the results of the workout. For instance, a doctor or therapist is usually limited in the ability to actually monitor the exercise or other therapy being performed by a user. There are existing devices that can be worn by a patient exercising at a given location, for instance at a doctor office or rehabilitation center. Likewise, there may be some exercise devices that can communicate with a central communication center at a doctor office or therapy center. These types of monitoring set ups are often very difficult and cumbersome to arrange.

Finally, existing devices do not readily allow a user to provide his or her own input or feelings that they have during the exercise or therapy that is being recommended to them. For instance, a device that monitors jogging or bicycle riding does not allow a user to comment about how they feel during the performance of that exercise. Subsequent recollection of feelings may not be as accurate as contemporaneous comments.

### SUMMARY OF THE INVENTION

Accordingly, it is an object of the present invention to overcome the foregoing drawbacks and limitations of known monitoring devices and to provide a device that monitors therapy or training through a prescribed exercise program. The device described herein may be programmed by the user or, alternatively, may be programmed by a trainer or by a therapist caring for the user. Further, the feedback provided by the device described herein may be both informational, motivational, supervisory or instructional. The specific interaction and substance of the interaction between the user and the monitoring device may also be customized.

In one embodiment, a physiological monitoring device includes a physiological detector, a controller and a feedback device. Each of these components is adapted to be attached to a user. The detector picks up information regarding a user's physiological condition and sends that information to the controller. The controller includes a set up interface for initiating an exercise and a storage device. The storage device stores a predetermined exercise program, receives and stores information from the detector, compares the information from the detector with the predetermined exercise program, and based on that comparison, generates messages for the user contemporaneously with the exercise. The feedback device delivers the messages to the user contemporaneously with the exercise. The predetermined exercise program can be customizable. The controller may include a port that allows the predetermined exercise program to be modified. The port may also allow the stored information from the detector to be downloaded. The monitoring device may further include a transmitter connected to the controller and adapted to transmit information from the controller. The port described herein may be connectable with a CPU. The CPU can be connectable to an Internet website that contains a plurality of predetermined exercise programs that are inputtable into the controller. The CPU is adapted to allow a user to customize the predetermined exercise program. Alternatively, the CPU can be adapted to process a computer disk containing a predetermined exercise program. The monitoring device setup interface can be voice-activated. Also, the controller may include a voice-activated, user interface.

In a further embodiment, an exercise monitoring device includes an exercise detector, a controller, and a feedback device. Each of these components is adapted to be attached to a user. The detector picks up information regarding a user's performance of an exercise and sends that information to the controller. The controller includes a setup interface for initiating an exercise and a storage device. The storage device stores a predetermined exercise program, receives and stores information from the detector, compares the information from the detector with the predetermined exercise program, and based on that comparison, generates messages for the user contemporaneously with the exercise. The feedback device delivers the messages to the user contemporaneously with the exercise. The predetermined exercise program can be customizable. The controller may include a port that allows the predetermined exercise program to be modified. The port may also allow the stored information from the detector to be downloaded. The monitoring device may further include a transmitter connected to the controller and adapted to transmit information from the controller. The port described herein may be connectable with a CPU. The CPU can be connectable to an Internet website that contains a plurality of predetermined exercise programs that are inputtable into the controller. The CPU is adapted to allow a user to customize the predetermined exercise program. Alternatively, the CPU can be adapted to process a computer disk containing a predetermined exercise program. The monitoring device setup interface can be voice-activated. Also, the controller may include a voice-activated, user interface.

### BRIEF DESCRIPTION OF THE DRAWINGS

FIG. 1 is a schematic view of a person wearing a heart rate monitor and headphones in accordance with one embodiment of the present invention.

FIGS. 2 and 2A are a top view and side view of a detector and controller housing in accordance with an embodiment of the present invention.

FIGS. 3 and 3A are side and top views respectively of a docking station to be used in connection with one embodiment of the present invention.

FIG. 4 is a flow chart diagram describing the operation of one embodiment of the present invention.

FIG. 5 is a flow chart describing a heart beat interval detection algorithm used in connection with a specific embodiment of the present invention described herein.

### DETAILED DESCRIPTION OF THE INVENTION

The invention is a convenient and portable device for use with exercise or physical therapy or physiological monitoring generally. The exercise program that is prompted and recorded in accordance with the present invention may be easily changed or modified by the user or by a third party such as a trainer or therapist. In accordance with the present invention, data obtained from the detector is stored for immediate feedback or for later downloaded review and analysis.

The specific example described in detail here is a heart rate monitor. Of course, other physiological functions may be monitored, including but not limited to, blood pressure, blood oxygen, alertness (brain waves), etc. Also, an exercise may itself be detected and monitored. Exercise functions that could be monitored include weight-lifting repetitions, distance measurements, resistance training, etc. In each of these alternative cases, the actual hardware would of course be different, particularly a detector, but all of the functionality of the components would be the same. A user would still enjoy the benefits of the variable and accountable system described herein.

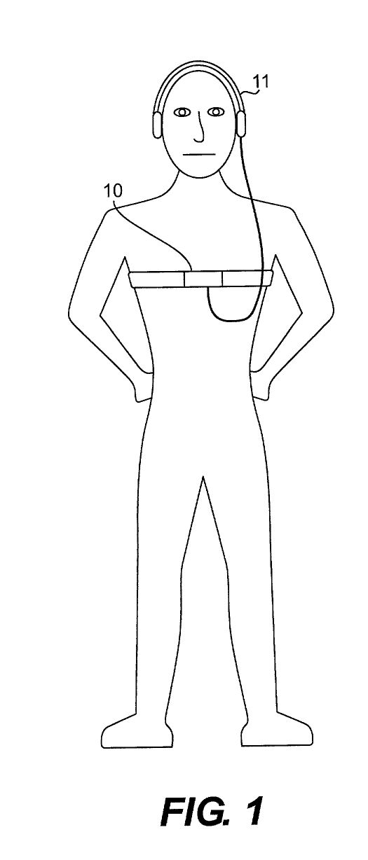

Turning now to FIG. 1, there is shown an illustrative example of a person wearing a chest strap heart rate monitor 10 and a set of headphones 11 worn by the user and plugged into the monitor 10.

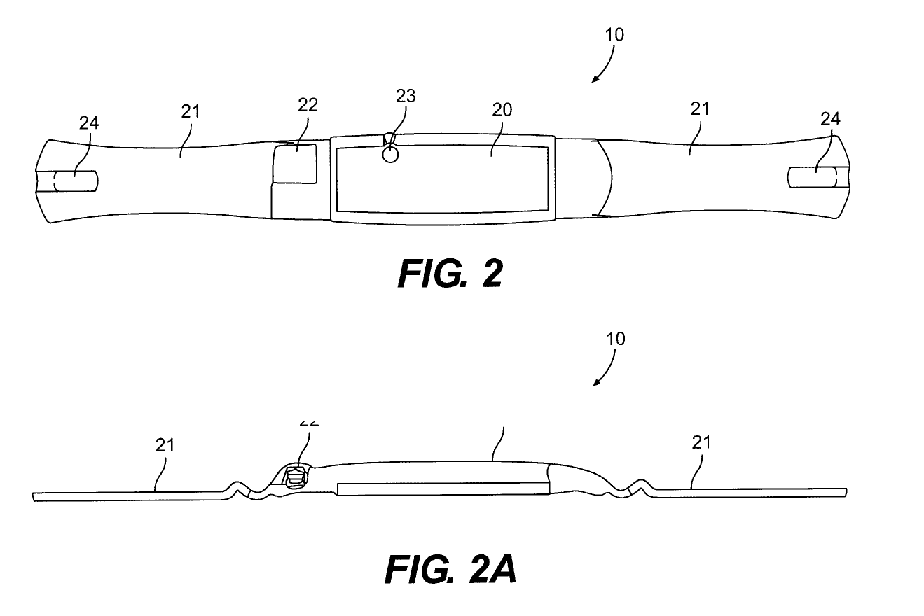

Turning now to FIGS. 2 and 2A, the monitor 10 is shown as being made up of a housing 20, electrodes 21, and a docking station/audio connector 22. The housing 20 further includes a button 23 that is the setup interface to be used by a person to initiate an exercise program. The electrodes 21 are the actual detectors that pick up the heart beat of the user. Specifically, the electrodes 21 pick up the electrical current created by the body during each pulse. In the illustrated example, the electrodes 21 are themselves flexible portions made up of a conductive polymer material-conductive urethane. Alternatively, electrodes may be imbedded into a flexible strap. The electrodes 21 have chest strap connectors 24 which are merely apertures that receive a strap end and allow the monitor 10 to be held around a person's chest. The strap (not shown) is any elastic or flexible member that holds the monitor 10 against the chest of the user. The electrodes 21 are connected to a controller that is contained within the housing 20.

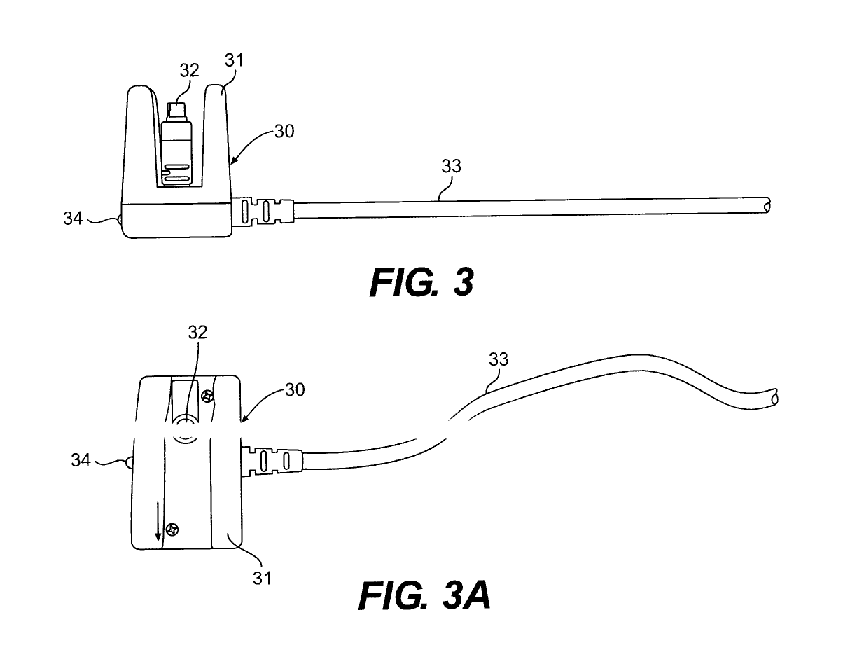

The docking station/audio connector 22 is a port that has at least two primary modes of operation. When the monitor 10 is in use by a user as shown in FIG. 1, the connector 22 receives the prong of the headphones 11. The connector 22 allows audio information from the controller in the housing 20 to be transmitted to the headphones 11 and, therefore, to the user. In the recharge and docking mode, the connector 22 is mounted onto a docking station (see FIGS. 3 and 3A) in order to charge a battery in the controller in the housing 20. Also, when the connector 22 is used in its docking mode, the controller 20 is able to be in communication with a CPU that allows the flow of information back and forth between the storage unit in the controller and the user or a third party. The user or third party may download information picked up by the detectors (electrodes 21) and modify the specific audio feedback (motivational and/or instructional script) when the monitor 10 is in use.

FIGS. 3 and 3A illustrate the docking station 30. The docking station 30 is made up of a cradle 31 adapted to receive the monitor 10. Specifically, the connector 32 of the docking station 30 is adapted to be inserted into the connector 22 on the monitor 10. The docking station 30 further includes a cable 33 allowing power and information to flow into the monitor 10. The power indicator 34 is a convenient light which a user may observe to insure that the monitor 10 is securely mounted into the docking station 30. Finally, the docking station 30 preferably incorporates a reset button (not shown) that allows an effective shutting down and restarting of the controller without losing either the data stored in the controller or the place in the operation of the current workout.

The docking station 30 may also incorporate circuitry that assists the download of information from the monitor 10. An intelligent circuit measures the current from the monitor 10. The circuit then compensates, if necessary, for a low current or no current (dead battery) in the monitor. When a communication is detected between a CPU and the Storage Device in the Controller, then the Docking Station will increase the current to the Storage Deve to ensure that the Storage Device has power to operate and communicate with the CPU. When there is no communication between the monitor and a CPU, then a conventional, low current for recharging a batter flows into the monitor. If there is no circuit to compensate for low/no power, then the information might not be available until the monitor battery was recharged.

The specific configuration of the docking station 30 also provides a safety advantage. The cradle 31 is a mechanical barrier to prevent a user from wearing the monitor 10 while it is in the dangerous mode of being connected to a power source. The cradle 31, therefore, prevents a potentially dangerous misuse of the monitor 10.

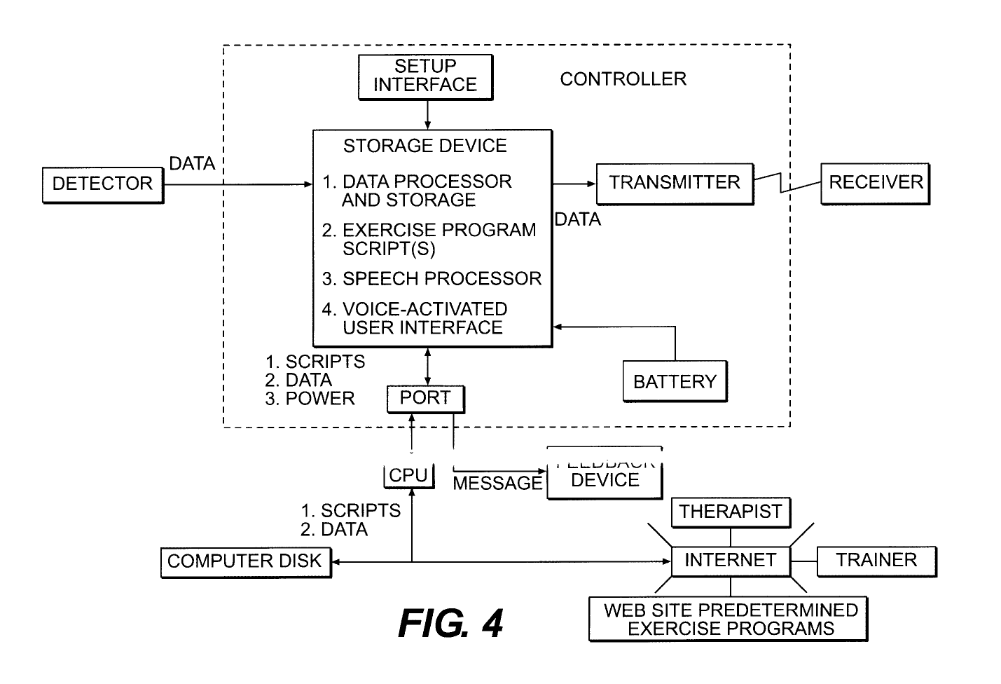

Turning now to FIG. 4, there is seen a flow chart of the operation of one embodiment of the monitor otherwise shown in FIGS. 1-3. For ease of understanding of the flow of information and functionality, the important terms have been spelled out in the flow chart. Examples of earlier patents that describe monitors with preset, unchangeable programs and that describe some hardware that could possibly be used to make up certain, specific portions (but not the complete structure) of the monitor discussed herein are as follows: U.S. Pat. No. 6,026,335 to Atlas, “Heart Rate Monitor With Age-Dependent Target-Zone Feedback,” issued Feb. 15, 2000; U.S. Pat. No. 5,857,939 to Kaufman, “Exercise Device With Audible Electronic Monitor,” issued Jan. 12, 1999; U.S. Pat. No. 6,251,048 to Kaufman, “Exercise Device with Audio Electronic Monitor,” issued Jun. 26, 2001; and any other related applications. These patents are incorporated by reference herein as if set forth in their entirety.

A Detector is used to pick up signals from the body relating to a physiological function of the body. In the specific example noted, the Detector is an electrode that is used to pick the electric waves in the body resulting from a heartbeat. The Detector specifically is able to pick up the ECG data and deliver that data to a processor designated as the Storage Device. The Storage Device is one component of the Controller that is found within the housing 20 shown in FIGS. 2 and 2A. The Detector is specifically shown as the electrodes 21 in FIGS. 2 and 2A. As noted earlier, a Detector may also monitor the performance of an exercise itself (e.g., count repetitions).

In FIG. 4, the Controller is made up of the following components: Setup Interface, Storage Device, Transmitter and Port. The Setup Interface is the button 23 shown in FIG. 2 that allows the user to manually initiate an exercise program or therapy. The Setup Interface is connected to the Storage Device. The Setup Interface may be voice or sound activated. There may alternatively be manual initiation (e.g., a push button) of a voice setup. There may even be the option of manual or voice setup. Structurally, a monitor would need a microphone to pick up voice signals and voice recognition software in a controller to process and interact with the voice signals. This type of equipment and software is conventionally available and known to those of skill in the art.

The Storage Device is a processor and memory component that stores the physiological information picked up by the Detector, in this case, the ECG information. As a matter of practicality in this particular example, the Storage Device must include other functional features that include evaluating the quality of the detected signal to make sure that the Detector is in fact in electrical contract with the body of the user. The Storage Device processor must also cull out any noise or inaccurate signals that result from mechanical or other intervening events not related to the physiological function that is desired to be monitored.

The Storage Device also includes a Speech Processor. The Speech Processor is the portion of memory that enables the Storage Device (controller) to send a signal that can be converted into audio output. The Speech Processor interacts with Exercise Program Scripts that are also contained in the Storage Device. The Exercise Program Scripts are the predetermined exercise program or programs that are programmed into the controller that are initiated by the user using the set up interface. The Exercise Program Script is used by the Storage Device to compare the data obtained from the detector with the information contained in the predetermined exercise program to then generate messages for the user contemporaneously with the exercise.

One processor that performs most if not all of the functions described herein is a Texas Instruments Speech Processor Chip, Model No. MSP50C605A. In addition to speech processing capabilities, the TI chip can store and process data from a detector. It may also be programmed with exercise program scripts. Importantly, the processor must be programmable in almost every aspect by a user or by a therapist or trainer working with a user. The language, the timing of the speech, the ability to interact with external CPUs to modify or change an exercise program are just some of the flexible features to be derived from the programmability of the processor. Of course, other processors could be adapted to work and fulfill the functions described herein.

The Port is the means by which scripts, data and power are allowed to flow into and out of the Controller. As shown in FIG. 2, the Port is a docking station/audio connector. This Port interacts with the docking station 30 shown in FIGS. 3 and 3A. As shown, the Port is a component that physically links the controller to a feedback device and to a CPU. This link may also be done through wireless means known to those of skill in the art. The Port would be a transmitter and receiver of information and data. (The storage device would still require a source of power, whether in the form of a battery or a rechargeable power source.) As shown, the connector 22 is an audio output connector when the headphones 11 are plugged into the monitor 10. In this way, the storage device can send information through the Port to the headphones (feedback device) to pass information to the user of the monitor. The alternative mode of operation of the port is to be plugged into the docking station 30. The docking station 30 is connected to a CPU that will typically be a personal computer owned and operated by the user. The CPU is in turn connected or connectable to the Internet. In this way, exercise program scripts as well as exercise data may be exchanged with, for instance, a therapist or trainer. Similarly, a user could go to a specific website or purchase a preprogrammed computer disk to obtain a predetermined exercise program to fit their training or monitoring needs. Finally, the user could themselves program an exercise script from the CPU directly into the storage device by means of the Port.

The Feedback Device is shown as a set of headphones 11 physically connected to the monitor 10. The connection may be accomplished with known wireless technology. Also, the “feedback” is discussed in terms of words and speech. For this invention, “feedback” may take other forms such as nonverbal sound, visual signals, or physical signals (e.g. vibration). Still further, “feedback” is not substantively limited to merely reporting physiological conditions or exercise results. The messages programmed into the controller and set to a user may also include motivation, instructional, supervisory or generally informative language. The message could include instructions about how to use a machine or perform an exercise. It may include custom language from a therapist, trainer or the user him/herself.

In a further alternative, the Controller includes a Transmitter. The Transmitter is a radio frequency transmitter that sends the physiological data or exercise information collected by the Detector to a Receiver. (The Transmitter could also operate by infrared, magnetic induction or other known transmission technologies.) Common exercise equipment that is available that incorporates a transmitter/receiver setup includes a heart rate monitor with a transmitter and a watch having a receiver so that a user can easily see their heart rate. Similarly, treadmills or bicycles may have receivers that allow a user to very conveniently observe their heart rate during use.

In a monitor containing a microphone (voice receiver) and voice recognition software as discussed earlier, the Storage Device may include further processing capabilities that allow a user to control an exercise machine through verbal commands. In other words, voice recognition software imbedded in or programmed into the Storage Device can convert a verbal command into a signal that is sent by the Transmitter to an exercise machine to control its operation. Again, the software and hardware to implement this feature are well known to those skilled in the art.

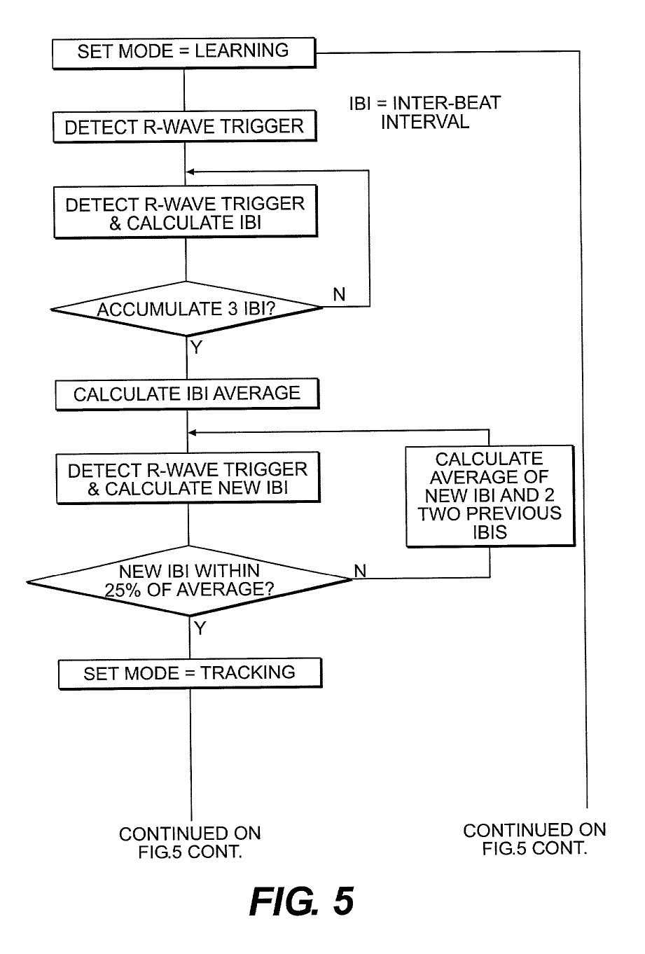

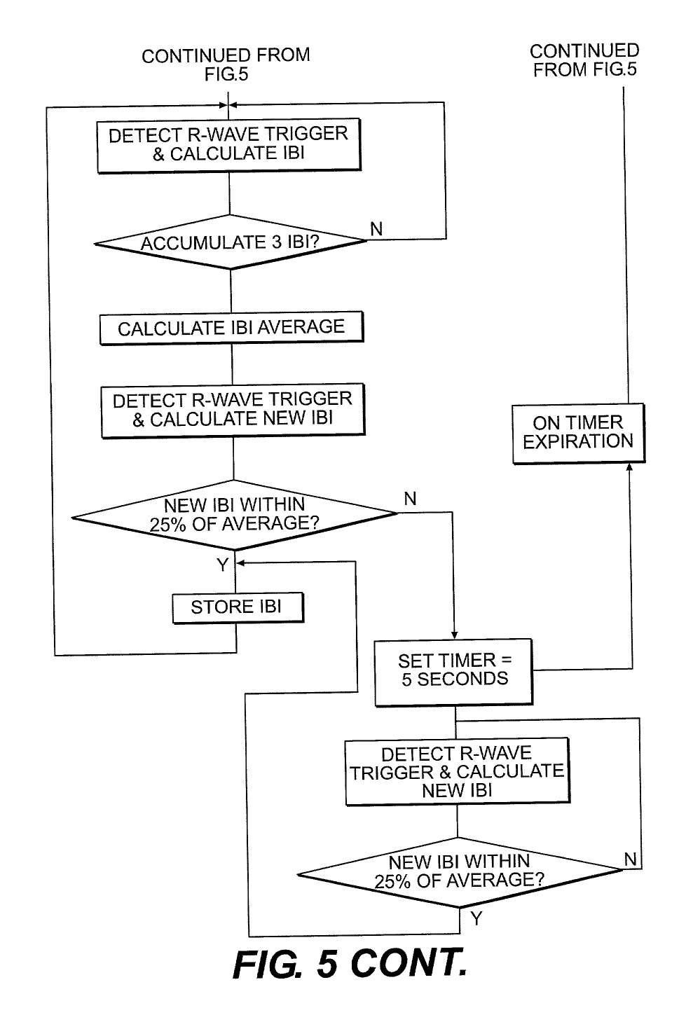

FIG. 5 shows the specific algorithm that may be used to calculate heart rate. Again, the flow chart includes the written flow of information that is used in detecting an accurate inter-beat interval. The algorithm demonstrates how the monitor decides the accurate heart beat while ignoring any noise signals or incomplete signals. Of course, those of skill in the art will be able to design similar algorithms to accurately arrive at a user's heart rate. Other algorithms will be relevant in collecting information from other types of detectors.

There are many variations in the use of the system described herein that render the described device as a very flexible device serving many purposes. First, a user may select any exercise program that they want to and have it input into the monitor to provide custom feedback, whether it is in the form motivational speech or warning speech, for instance. The exercise program may be input by the trainer or therapist of a user. For instance, a patient recovering from heart disease may need a specific regimen to best make them healthy. The described device allows for the customization of a workout. There may further be a website that a user may go to to download a desirable exercise program in the form of a script. For instance, a website might have a training regimen for a marathoner or a triathlete or a heart attack victim in recovery. Different scripts may be developed for broad use and offered for sale over the Internet at that website. Other athletes could exchange scripts over the Internet. Other scripts may be made available on computer disks (cd's, zip disks, etc.) that a user could buy and download into a CPU for loading into the device.

Another important aspect of the described device is the monitoring and accountability feature of it. In addition to customizing the workout, the monitor records and stores the results of the workout. For instance, a therapist can download the data obtained from a patient to determine how well they performed an exercise and how to best determine a future exercise program for an individual. A therapist or trainer can also customize the verbal instruction or audio feedback to a patient in order to best support or motivate a user. With respect to accountability, the user cannot “fake” an exercise. The downloaded information would easily expose an individual not following a workout.

There are also safety features as a result of the present device. Important data that is picked up during an exercise may be stored and downloaded. Also, in the event of a dangerous physical condition (high heart rate) that develops during exercise, the monitor can instruct a user to rest or take other precautions. Further, in a monitor that has a transmitter, a distress signal may be generated and sent in the event of a catastrophic event.

Finally, a monitor may include a recording device. The recording device would be connected to the controller and synchronized with the controller. A user could then provide real time comments about a particular exercise or work out. This could facilitate a trainer or therapist with respect to formulating future exercise. A microphone would be connected to the monitor, and a recorder would be incorporated into the Controller. Some type of manual switch or button would activate the recorder, or the particular exercise program may activate the recorder seeking feedback from the user.

While the invention has been described with reference to specific embodiments thereof, it will be understood that numerous variations, modifications and additional embodiments are possible, and accordingly, all such variations, modifications, and embodiments are to be regarded as being within the spirit and scope of the invention.

In the claims:

1. A physiological monitoring device comprising:
a physiological detector, a controller and a feedback device, each adapted to be attached to a user;
wherein the detector picks up information regarding a user's physiological condition and sends that information to the controller;
wherein the controller comprises a setup interface for initiating an exercise, and a storage device for
a) storing a predetermined exercise program,
b) receiving and storing information from the detector,
c) comparing the information from the detector with the predetermined exercise program, and
d) based on the comparison, generating messages for the user contemporaneously with the exercise;
and further wherein the feedback device delivers the messages to the user contemporaneously with the exercise.

2. A physiological monitoring device described in claim 1, wherein in the predetermined exercise program is customizable.

3. A physiological monitoring device as described in claim 1, wherein the controller further comprises a port that allows the predetermined exercise program to be modified.

4. A physiological monitoring device as described in claim 1, wherein the controller further comprises a port that allows the stored information from the detector to be downloaded.

5. A physiological monitoring device as described in claim 3, wherein the port also allows the stored information from the detector to be downloaded.

6. A physiological monitoring device as described in claim 1, further comprising a transmitter connected to the controller and adapted to transmit information from the controller.

7. A physiological monitoring device as described in claim 3, further wherein the port is connectable with a CPU.

8. A physiological monitoring device as described in claim 4, further wherein the port is connectable with a CPU.

9. A physiological monitoring device as described in claim 5, further wherein the port is connectable with a CPU.

10. A physiological monitoring device as described in claim 7, further comprising an internet web site that is connectable to the CPU and that contains a plurality of predetermined exercise programs that are inputtable into the controller.

11. A physiological monitoring device as described in claim 7, wherein the CPU is adapted to allow a user to customize the predetermined exercise program.

12. A physiological monitoring device as described in claim 7, wherein the CPU is adapted to process a computer disk containing a predetermined exercise program.

13. A physiological monitoring device as described in claim 1, wherein the setup interface is voice-activated.

14. A physiological monitoring device as described in claim 1, wherein the controller further comprises a voice-activated, user interface.

15. A physiological monitoring device as described in claim 6, wherein the controller further comprises a voice-activated, user interface.

16. An exercise monitoring device comprising:
an exercise detector, a controller and a feedback device, each adapted to be attached to a user;
wherein the detector picks up information regarding a user's performance of an exercise and sends that information to the controller;
wherein the controller comprises a setup interface for initiating an exercise, and a storage device for
a) storing a predetermined exercise program,
b) receiving and storing information from the detector,
c) comparing the information from the detector with the predetermined exercise program, and
d) based on the comparison, generating messages for the user contemporaneously with the exercise;
and further wherein the feedback device delivers the messages to the user contemporaneously with the exercise.

17. An exercise monitoring device as described in claim 16, wherein in the predetermined exercise program is customizable.

18. An exercise monitoring device as described in claim 16, wherein the controller further comprises a port that allows the predetermined exercise program to be modified.

19. An exercise monitoring device as described in claim 16, wherein the controller further comprises a port that allows the stored information from the detector to be downloaded.

20. An exercise monitoring device as described in claim 18, wherein the port also allows the stored information from the detector to be downloaded.

21. An exercise monitoring device as described in claim 16, further comprising a transmitter connected to the controller and adapted to transmit information from the controller.

22. An exercise monitoring device as described in claim 18, further wherein the port is connectable with a CPU.

23. An exercise monitoring device as described in claim 19, further wherein the port is connectable with a CPU.

24. An exercise monitoring device as described in claim 20, further wherein the port is connectable with a CPU.

25. An exercise monitoring device as described in claim 22, further comprising an internet web site that is connectable to the CPU and that contains a plurality of predetermined exercise programs that are inputtable into the controller.

26. An exercise monitoring device as described in claim 22, wherein the CPU is adapted to allow a user to customize the predetermined exercise program.

27. An exercise monitoring device as described in claim 22, wherein the CPU is adapted to process a computer disk containing a predetermined exercise program.

28. An exercise monitoring device as described in claim 21, wherein the setup interface is voice-activated.

29. An exercise monitoring device as described in claim 16, wherein the controller further comprises a voice-activated, user interface.

30. An exercise monitoring device as described in claim 21, wherein the controller further comprises a voice-activated, user interface
